(19) United States
(12) Patent Application Publication
(10) Pub. No.: US 2024/0382500 A1
(43) Pub. Date: Nov. 21, 2024

(54) DOPAMINE RECEPTOR AGONISTS OR ANGIOTENSIN RECEPTOR BLOCKERS OR COMBINATIONS THEREOF FOR COVID-19 SEQUELAE

(71) Applicant: Adonis Sfera, Redlands, CA (US)

(72) Inventor: Adonis Sfera, Redlands, CA (US)

(21) Appl. No.: 18/666,924

(22) Filed: May 17, 2024

Related U.S. Application Data
(60) Provisional application No. 63/503,122, filed on May 18, 2023.

(51) Int. Cl. A61K 31/55; A61K 31/4184; A61K 31/506

(52) U.S. Cl. CPC A61K 31/55; A61K 31/4184; A61K 31/506

(57) ABSTRACT

Methods and compositions are disclosed to treat acute COVID-19 or sequelae of acute COVID-19 infection, commonly called “long COVID”. Two therapeutic agents, a selective dopamine receptor 1 (D₁R) partial agonist, and a blood-brain barrier (BBB)-crossing angiotensin receptor blocker (ARB), administered in combination, are expected to ameliorate the effects of premature cellular senescence, enhance efferocytosis, decrease the permeability of the gut barrier to pathogens and/or their components, eliminate virus-infected senescent cells, avert cell-cell fusion, and/or avert fusion-induced senescence (FIS). These effects in turn are expected to treat acute COVID-19 infection and reverse “long COVID” by eliminating reservoirs of SARS-COV-2 virus that cause symptomatic sequelae.

### FIELD OF THE INVENTION

This disclosure relates to methods and compositions to treat sequelae from acute COVID-19 infection. The methods and compositions eliminate persistent SARS-COV-2 viral reservoirs from senescent cells that may cause sequelae from acute COVID-19 infection, with the use of a selective dopamine receptor 1 (D₁R) partial agonist in combination with a blood-brain barrier (BBB)-crossing angiotensin receptor blocker (ARB).

### BACKGROUND

Like previous pandemics, COVID-19 has been succeeded by well-documented post-infectious sequelae, colloquially known as “long COVID.” This syndrome, manifested by chronic fatigue, cough, shortness of breath, myalgia, and concentration difficulties, may last for many months after the acute phase of illness. Long COVID affects all aspects of a patient's life, including work, rest, and activities of daily living, placing a large financial burden not only on the individual, but also on families, and the society at large.

Like other viruses, including Human Immunodeficiency Virus (HIV), SARS-COV-2 (the causative agent of COVID-19) may thrive in viral reservoirs, likely senescent cells,[1] including prematurely aged macrophages/microglia, that shield the virus from neutralizing antibodies, maintaining a low-grade infection documented in long COVID.[2] Since senescent cells upregulate angiotensin converting enzyme 2 (ACE-2), the SARS-CoV-2 entry portal, they may facilitate viral ingress and accumulation in these cells.[3] Vaccines exist to prevent SARS-COV-2 infection, and drug therapies exist to treat acute infection. There remains a need for new therapies to treat long COVID.

### SUMMARY OF THE INVENTION

This invention discloses a method for treating acute COVID-19 infection and sequelae of acute COVID-19 infection in a patient, including administering to the patient an effective amount of a selective dopamine receptor 1 (D₁R) partial agonist in combination with an angiotensin receptor blocker (ARB) that crosses the blood-brain barrier. This combination is expected to block entry of the SARS-COV-2 virus into cells that go on to act as cellular reservoirs and will also neutralize SARS-COV-2 viral particles within cells.

In an embodiment, the D₁R partial agonist is selected from at least one of the group consisting of SKF38393, SKF75670, SKF77434, fenoldopam, SKF83959, chloro-APB, SKF81297, SKF82957, SKF83822, SCH-39166, PF-4211, PF-8294, PF-2334, PF-6142, PF-1119, PF-06649751, and ecopipam. In an embodiment, the D₁R agonist is SKF38393 or PF-06649751.

In an embodiment, the ARB is candesartan or telmisartan.

In an embodiment, a combination of a D₁R partial agonist and an ARB may be administered in a unit dosage form for oral administration. In an embodiment, the unit dosage form may be selected from the group consisting of a tablet, a capsule, a liquid for oral administration, and a powder.

In an embodiment, a kit is provided having separate oral dosage forms of a D₁R partial agonist and an ARB for administration to the patient. Such a kit can provide for a non-unit dose of the drug or for non-sequential administration of a combination of a D₁R partial agonist and an ARB.

A composition for treating sequelae of acute COVID-19 infection in a patient, comprising a unit dosage form containing a selective D₁R partial agonist and an angiotensin receptor blocker (ARB) that crosses the blood-brain barrier, or a combination thereof.

In an embodiment, the D₁R partial agonist in the composition is selected from at least one of the group consisting of SKF38393, SKF75670, SKF77434, fenoldopam, SKF83959, chloro-APB, SKF81297, SKF82957, SKF83822, SCH-39166, PF-4211, PF-8294, PF-2334, PF-6142, PF-1119 and PF-06649751. In an embodiment, the D₁R partial agonist is SKF38393 or PF-06649751. In an embodiment, the ARB in the composition is candesartan or telmisartan.

The composition of claim 13, wherein the unit dosage form is selected from the group consisting of a tablet, a capsule, and a powder suitable for dissolving in a liquid.

The composition of claim 13, further comprising an excipient selected from at least one of the group consisting of binders, fillers, disintegrants, glidants, lubricants and flavoring agents, or a combination thereof.

### DESCRIPTION OF THE FIGURES

FIG. 1 Exogenous or endogenous viruses can drive cell-cell fusion or syncytia formation, generating giant multinucleated cells. Fusion starts with a fusion pore in the cell membrane that triggers Ca²⁺ influx, activating TMEM16F, a scramblase that promotes PS externalization, marking the cell ready for apoptosis or fusion. Fused cells trigger premature cellular senescence, known as fusion-induced senescence (FIS). Syncytial structures are resistant to elimination therefore, capable of harboring the virus for a long time, comprising an ideal viral reservoir. Taken from [4].

FIG. 2 SARS-COV-2 upregulates angiotensin II (ANG II), disrupting efferocytosis. This in return alters the permeability of gut barrier and BBB, allowing microbial translocation outside of the GI tract. Translocated microbes and/or their components trigger further senescence, especially in the cells of biological barriers. Fatiguing illnesses, including ME/CSF, CRF, and long COVID have been associated with premature cellular senescence and microbial translocation. Both candesartan and SKF38393 inhibit angiotensin II type 1 receptors (AT₁Rs), lowering the effects of excessive ANG II thus, correcting efferocytosis and barrier dysfunction. Taken from [5].

### DETAILED DESCRIPTION

Two therapeutic agents, a selective dopamine receptor 1 (D₁R) partial agonist, and a blood-brain barrier (BBB)-crossing angiotensin receptor blocker (ARB), administered in combination, are expected to eliminate virus-infected senescent cells, avert cell-cell fusion and avert fusion-induced senescence (FIS). These effects in turn are expected to eliminate reservoirs of SARS-COV-2 virus during acute infection and lingering after acute COVID-19 infection causing symptomatic sequelae, thereby reversing “long COVID.”

In an embodiment, the D₁R agonist is SKF38393, and the ARB is candesartan or telmisartan. SKF3839 [6] and candesartan [7], [8] both exhibit anti-inflammatory and pro-cognitive effects as well as decreasing senescent cells' accumulation by enhancing efferocytosis (clearance of senescent, virus-infected cells, via natural killer cells (NKCs) and macrophages/microglia).[9]

The D₁R partial agonists and ARB combinations of this invention are administered in a safe dosage effective to ameliorate the effects of premature cellular senescence, enhance efferocytosis, and/or decrease the permeability of the gut barrier to pathogens and/or their components.

### Selective Dopamine Receptor 1 (D₁R) Partial Agonists

The D₁R is employed by HIV to gain entry in cells.[10] Under the assumption that SARS-COV-2 has many similarities to HIV, D₁R's likewise mediate entry of SARS-COV-2 into cells including microglia and astrocytes.[11],[12] D₁R partial agonist activity may be desirable in this invention to block viral entry into cells. The D₁R active compounds discussed below show both agonist and agonist activity and are therefore termed “partial agonists.”

Several D₁R active compounds such as SKF38393 may be useful in this invention to block SARS-COV-2 entry in neuronal cells. Other D₁R partial agonists that may be of value in this invention include SKF75670, SKF77434, SKF83959, and SKF82957 PF8294, PF6142, PF-06649751 and others.[13],[14] When tested as D₁R agonists, these compounds show potent inhibition of dopamine-induced β-arrestin recruitment and D₁R internalization.[13] β-arrestins mediate dopamine receptor signaling[15] and the desensitization of G protein-coupled receptors (GPCR) signaling and therefore serve to “turn off” signaling, resulting in negative feedback of G-protein-dependent GPCR signaling.[13] The drug ecopipam (PubChem CID 107930) a selective D₁-like receptor antagonist similar to SKF38393, has also been studied for the treatment of tourette syndrome in children as a selective dopamine receptor 1-like antagonist,[16],[17] and may similarly be useful in this invention. Structures are shown below with EC₅₀ data and efficacy (Eₘₐₓ) data. Eₘₐₓ is the maximum effect which can be expected from this drug (i.e. when this magnitude of effect is reached, increasing the dose will not produce a greater magnitude of effect).

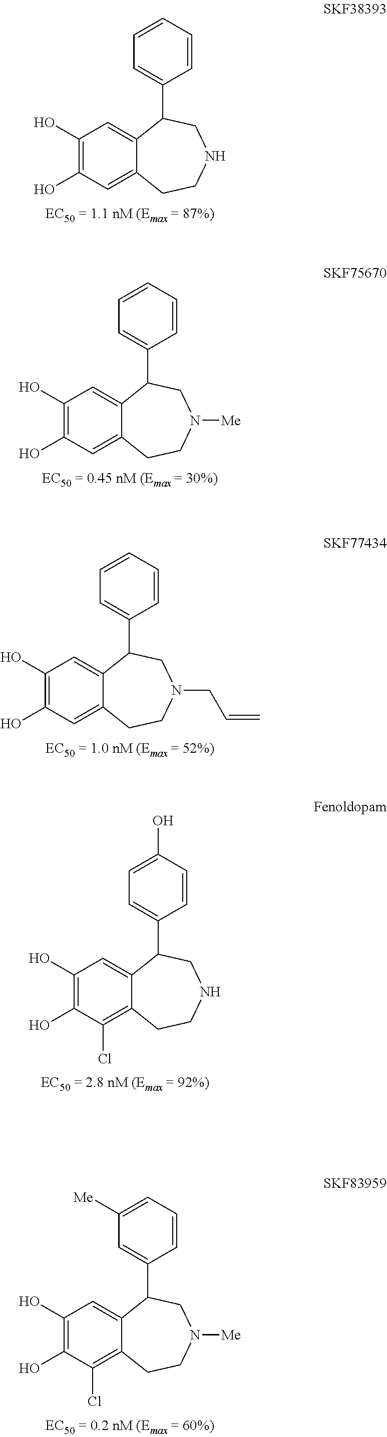 [SMILES: Oc1cc2c(cc1O)C(c1ccccc1)CNCC2 ; CN1CCc2cc(O)c(O)cc2C(c2ccccc2)C1 ; C=CCN1CCc2cc(O)c(O)cc2C(c2ccccc2)C1 ; Oc1ccc(C2CNCCc3c2cc(O)c(O)c3Cl)cc1 ; Cc1cccc(C2CN(C)CCc3c2cc(O)c(O)c3Cl)c1]

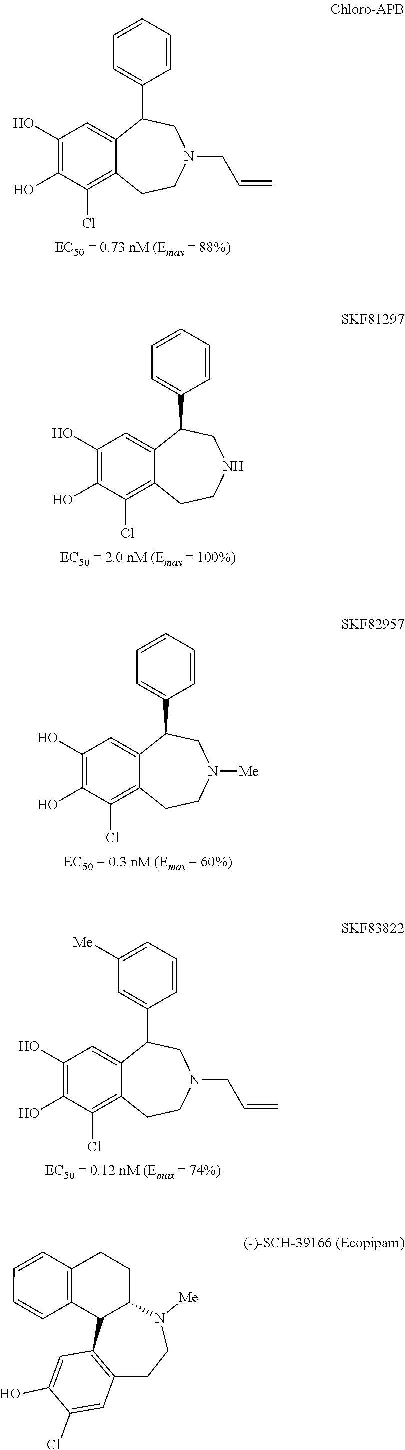

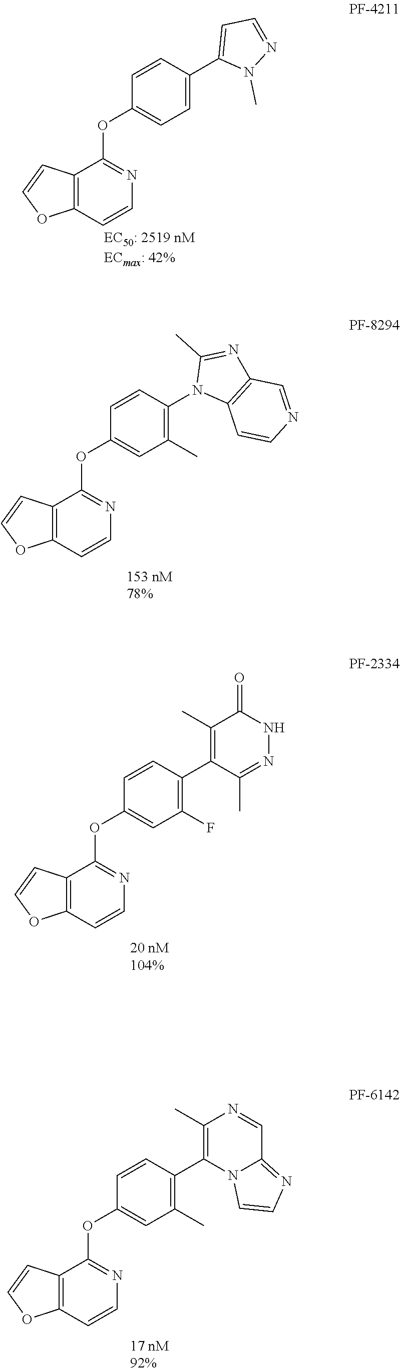 [SMILES: Cn1nccc1-c1ccc(Oc2nccc3occc23)cc1 ; Cc1cc(Oc2nccc3occc23)ccc1-n1c(C)nc2cnccc21 ; Cc1n[nH]c(=O)c(C)c1-c1ccc(Oc2nccc3occc23)cc1F ; Cc1cc(Oc2nccc3occc23)ccc1-c1c(C)ncc2nccn12]

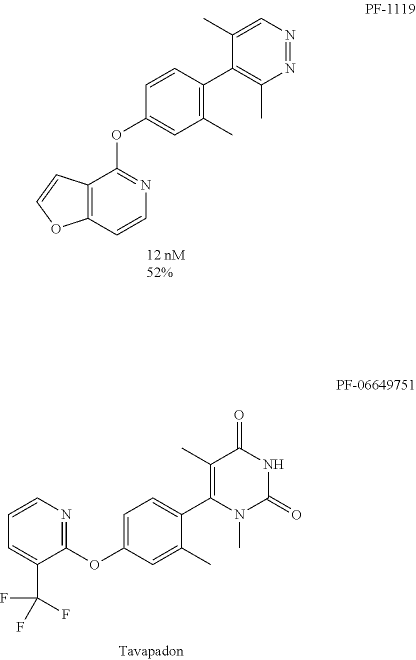 [SMILES: Cc1cc(Oc2nccc3occc23)ccc1-c1c(C)cnnc1C ; Cc1cc(Oc2ncccc2C(F)(F)F)ccc1-c1c(C)c(=O)[nH]c(=O)n1C]

D₁R is the most abundant dopamine receptor in both the rodent and human brain and its dysfunction was associated with many pathologies.[18] Several selective dopamine D₁R agonists and partial agonists have been developed for the treatment of Parkinson's disease, stimulant use disorders, and cognitive disorders.[19]-[21] Compared to newer agents, SKF38393 is a prototypical D₁R selective partial agonist on which a treasure trove of data has been available. Newer D₁R agonists may have similar effects however, their action on immune and senescent cells is poorly defined at this time.

In preclinical studies, SKF38393 was well tolerated and adverse effects were similar to those of levodopa. A noncatecholamine newer D₁R agonist, PF-06649751(Tavapadon) which is under active investigation for Parkinson's disease, was tested on human subjects in Phase I clinical trials and found to be safe and well tolerated (NCT02262767), (NCT02847650), (NCT01981694).[22]

The D₁R partial agonists of this invention are administered in a safe dosage effective to ameliorate the effects of premature cellular senescence, enhance efferocytosis, and/or decrease the permeability of the gut barrier to pathogens and/or their components.

### Angiotensin Receptor Blockers (ARB)

An important putative cellular reservoir of SARS-COV-2 is believed to be microglial cells. Such infected microglial cells may be neurotoxic and cause damage to healthy neurons. Brain organoids infected with SARS-COV-2 display disruption in circuit integrity via microglia-mediated synapse elimination.[23] ARB's are expected to neutralize these viral reservoirs by inhibiting angiotensin II (discussed more fully below). Because microglia are behind the BBB, a BBB-crossing ARB is desirable in this invention.

Candesartan may be a preferred ARB in this invention due its greater potency and selectivity compared to other approved ARB's, and its ability to cross BBB.[24] Telmisartan similarly crosses the blood brain barrier. Other approved ARB's, including olmesartan, eprosartan, irbesartan, and losartan do not cross the BBB.

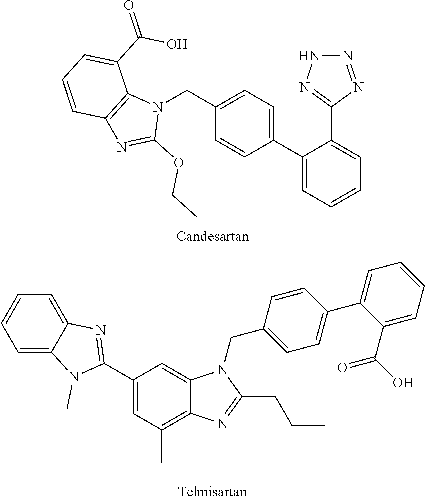 [SMILES: CCOc1nc2cccc(C(=O)O)c2n1Cc1ccc(-c2ccccc2-c2nn[nH]n2)cc1 ; CCCc1nc2c(C)cc(-c3nc4ccccc4n3C)cc2n1Cc1ccc(-c2ccccc2C(=O)O)cc1]

Candesartan is a synthetic, benzimidazole-derived angiotensin II receptor antagonist, a prodrug with antihypertensive activity. Candesartan selectively competes with angiotensin II (ANG II) for the binding of angiotensin II type 1 receptors (AT₁Rs) in vascular smooth muscle, blocking ANG II-mediated vasoconstriction and inducing vasodilatation. In addition, antagonism of AT₁R in the adrenal gland inhibits ANG II-stimulated aldosterone synthesis and secretion by the adrenal cortex. As a result, sodium and water excretion increase, followed by a reduction in plasma volume and blood pressure.

Candesartan is marketed as the cyclohexyl 1-hydroxyethyl carbonate (cilexetil) ester, known as candesartan cilexetil, i.e., a prodrug form of the drug. The use of a prodrug form increases the bioavailability of candesartan. Candesartan cilexetil is metabolised completely by esterases in the intestinal wall during absorption to the active candesartan moiety. In the first step of the cascading pro-drug mechanism, the carbonate group is hydrolyzed, releasing carbon dioxide. The metabolite at this step is cyclohexanol which, being relatively non-toxic, is advantageous to the design of the drug. The other aspect of the cascading prodrug is the O—CH—CH₃ moiety which becomes converted into acetic acid, which is another product from the cascading side reaction. Telmisartan is not marketed as a prodrug.

### The Consequences of Virus Induced Senescence (VIS)

The SARS-COV-2 virus is believed to induce premature cellular senescence and thrives in apoptosis-resistant, aging cells that become persistent viral reservoirs, which may account for the symptoms of long COVID.

Virus induced senescence (VIS) is an infection-mediated pathology driven by two mechanisms: (1) merging host cells into multinucleate syncytia that trigger a phenomenon known as fusion-induced senescence (FIS); and/or (2) upregulating cortisol and the high-mobility group box 1 (HMGB1), molecules which disrupt efferocytosis, causing accumulation of senescent, virus-infected, cells.

Senescent cells are resistant to apoptosis[25] and may act as viral reservoirs which can maintain a latent infection, explaining the symptoms of long COVID.

In the gut, senescent intestinal and endothelial cells (ECs) increase permeability, enabling translocation of microbes and/or their components into the systemic circulation. This in turn promotes further senescence, causing a vicious circle. For example, cellular senescence induced by lipopolysaccharide (LPS), a Gram-negative microbial molecule, has been well-documented.[26]

In contrast, an ARB such as candesartan and a D₁R agonist such as SKF38393,in combination, are expected to (1) ameliorate the effects of premature cellular senescence (2) enhance efferocytosis, and (3) decrease the permeability of the gut barrier to pathogens and/or their components. This hypothesis is supported by the following data:

(a) Several studies have documented that the SARS-COV-2 virus, akin to its viral counterparts, has the capability to induce cellular senescence (VIS), thereby creating a conducive environment for the replication and proliferation of viral progeny within the host cells.[25],[27] This phenomenon underscores the interplay between viral infection and cellular aging processes.[28],[29] Understanding these mechanisms is of paramount importance in devising effective therapeutic interventions and preventive strategies aimed at mitigating the impact of viral infections on human health.

(b) Fusion-induced senescence (FIS) is the ability of viruses, including SARS-CoV-2, to fuse host cells, prompting premature cellular senescence. This phenomenon has been elucidated in studies shedding light on the underlying mechanisms and consequences of FIS.[31] Additionally, specific viral agents, such as the measles virus, may induce cellular senescence through fusion events.[32] These findings emphasize the physiological and pathological ramifications of cellular senescence, accentuating the significance of understanding FIS in viral infections like COVID-19.[33]

(c) Previous studies have associated translocated intestinal microbes with Myalgic Encephalomyelitis/Chronic Fatigue Syndrome (ME/CFS), a condition marked by cellular senescence and a clinical picture similar to that of long COVID.[34]-[36]

(d) Cancer-related fatigue (CRF), a similar syndrome associated with chemotherapy or radiation-induced cellular senescence, is characterized by symptoms indistinguishable from those of long COVID.[37]-[40]

(e) Candesartan has anticancer properties as a potent inhibitor of the angiotensin II type 1 receptor (AT₁R).[41] The AT₁ receptor is implicated in tumor vascularization and angiogenesis.[42] Thus, inhibition of this receptor is anticipated to have anticancer effects. Candesartan is the most potent and selective of several ARB's currently available.[41] Additionally, candesartan crosses the blood brain barrier[24] and therefore is expected to exert inhibition of AT₁R on microglia cells, which are behind the BBB and are potential reservoirs of SARS-COV-2 virus causing long COVID.

(f) D₁R is upregulated in many cancer phenotypes.[43],[44] Thus, drugs such as SKF38393 is expected to have anticancer activity. Additionally, dopamine imbalance is implicated in fatigue and CRF.[45]

(g) ME/CFS, CRF and long COVID have been associated with the unchecked accumulation of senescent cells due to dysfunctional macrophages/microglia and NKCs, the key efferocytosis executors.[46]-[49]

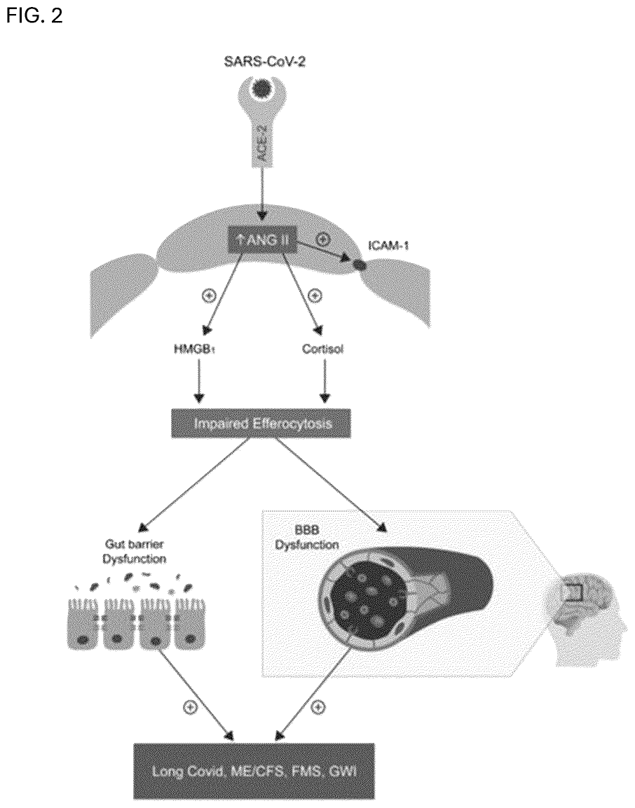

(h) Both psychological and biological stressors are known for increasing blood cortisol and HMGB1, biomolecules which directly impair efferocytosis (FIG. 2).[50],[51]

(i) HMGB1 and cortisol are associated with inflammatory bowel disease (IBD), a condition marked by significant fatigue, which is associated with dysfunctional gut barrier and microbial translocation.[52]

(j) Previous studies have associated the accumulation of senescent cells with inflammation and disruption of gut barrier and BBB, allowing microbes to migrate from the gastrointestinal (GI) tract into host tissues and organs, including the brain.[53],[54]

(k) NKCs and macrophage/microglia express viable dopaminergic and renin angiotensin systems (RAS), suggesting that candesartan and SKF38393 may directly enhance efferocytosis, lowering the abundance of senescent cells.[55],[56]

(l) Previous studies have shown that SKF38393 augments NKC cytotoxicity, while candesartan strengthens microglial phagocytosis, facilitating the elimination of senescent cells.[57],[58]

(m) The D₁R is believed to facilitate the entry of HIV into cells.[10] SARS-COV-2has many features in common with HIV, and D₁R's are implicated in the entry of SARS-CoV-2 into cells including microglia and astrocytes.[11],[12] Accordingly, blocking D₁R's is expected to block SARS-COV-2 entry into cells.

### Viral Reservoirs

Viral reservoirs are cellular types or phenotypes and tissue sites in which viruses can accumulate, replicate, and thrive after the acute phase of illness. For example, microglia and memory CD4(+) T cells are well-known HIV reservoirs in which the pathogen can persist in a latent state, averting exposure to the highly active antiretroviral therapy (HAART).[59] Other known viruses that can maintain latency in humans are herpes simplex virus (HSV), varicella zoster virus (VZV), and Epstein-Barr virus (EBV). For example, VZV can thrive in sensory-nerve ganglia, reservoirs from which it can be reactivated later in life.[59]

It is hypothesized here that the SARS-COV-2 virus induces cellular senescence and maintains latency in senescent cells which are metabolically active and long-lived (persistent) as they resist apoptosis as well as other modalities of programmed cell death, including ferroptosis.[60] In addition, senescent cells upregulate iron, a biometal necessary for viral replication as well as calcium (Ca²⁺) which plays a key role in virion formation, and translation of viral proteins.[60] Senescent macrophages, likely used by SARS-COV-2 as reservoirs, upregulate fibrogenesis, facilitating fibrosis, a key pathology of this virus.[61],[62]

Moreover, SARS-COV-2 has been shown to directly infect human microglia, the central nervous system (CNS) macrophages, inducing dystrophy or senescence, that may convert these cells into neurotoxic phenotypes, explaining the neuropsychiatric sequelae of long COVID.[63] Because microglia are behind the BBB, BBB penetrating drugs (such as candesartan, telmistartan, and the D₁R agonists disclosed herein) are important to attack and clear these reservoirs.

Taken together, the SARS-COV-2 virus promotes premature cellular, including macrophage/microglia, senescence, and thrives in a latent state, inducing low-grade inflammation, a likely driver of long COVID.

### Cellular Senescence and Biological Barriers

Cellular senescence is a physiological effect which protects cells against malignant transformation by arresting replication in response to exogenous or endogenous insults[64],[65] Senescent cells are metabolically active and release a detrimental secretome known as senescence-associated secretory phenotype (SASP) which can induce senescence in neighboring healthy cells, spreading both senescence and viral infection. Senescent cells upregulate intracellular iron and Ca²⁺, generating an ideal milieu for intracellular pathogens, including the SARS-COV-2 virus.[64],[66] For this reason, the virus not only promotes cellular senescence but may also “hide” in senescent cells, evading the neutralizing antibodies.[28],[67],[68] SARS-COV-2 has been implicated specifically in dopaminergic neuron senescence.[69] In addition, senescent cells exhibit shorter telomeres and overexpress ACE-2, the SARS-COV-2entry portal, facilitating viral ingress, likely explaining the high risk of COVID-19complications in older individuals.[70] This also explains the reason senolytic agents are believed beneficial for both COVID-19 and long COVID.[27],[35] Although D₁R agonists and ARB's have not previously been conceptualized as senolytic drugs, they ameliorate the effects of ECs senescence and lower SASP, suggesting senolytic properties.[27],[71]

### COVID-19 and Fusion-Induced Senescence (FIS)

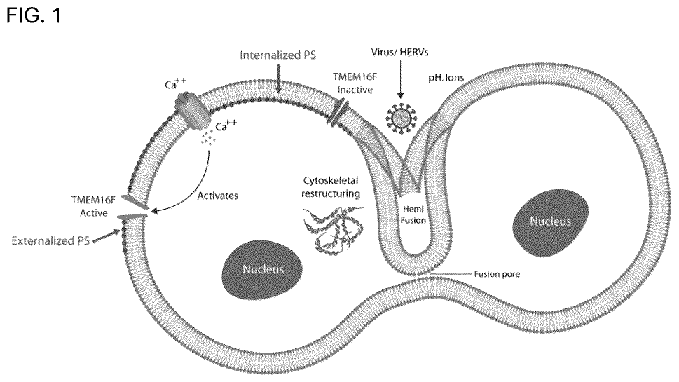

COVID-19 can induce premature cellular senescence in host cells by generating syncytial structures, multinucleated giant cells, resistant to apoptosis or efferocytosis (FIG. 1). For example, multinucleated cancer giant cells, generated by ionizing radiation, were demonstrated to retain viability and resist elimination.

The SARS-COV-2 spike protein (S protein) contains arginine-rich cell-penetrating peptides which drive cell-cell fusion and the subsequent FIS.[31]-[33] (FIG. 1). The SARS-COV-2 virus can also activate human endogenous retroviruses (HERVs) that in return trigger fusion of host cells, forming syncytia in which the pathogen can thrive (FIG. 1).[73], [74]

HERVs are viral fossils, comprising about 8-9% of the human genome, which have originated with ancient viral infections and were incorporated into the DNA. Some HERVs have been “domesticated” and have assumed physiological functions

(HERV-W env playing a major role in placentation).[76] For example, HERVs express several fusogens, including syncytins, which enable the formation of placental syncytiotrophoblast, and can be exploited by viruses to generate FIS.[77] Exogenous viruses, including SARS-COV-2, have been demonstrated to activate HERVs, probably triggering FIS, a pathology documented in ME/CFS and long COVID.[78]-[80]

The process of cell-cell fusion requires activation of a molecular machinery that drives cytoskeletal remodeling to form giant cells. This molecular apparatus is comprised of TMEM16F, a calcium-dependent scramblase, that flips phosphatidylserine (PS) from the inner leaflet of cell membrane to the cell surface where it signals readiness for apoptosis or fusion (FIG. 1).[81] A Ca²⁺/calmodulin-dependent protein kinase II (CaMKII) system was identified in the S protein of the SARS-COV-2 virus, suggesting that TMEM16F may be activated by the Ca²⁺ influx alone, without the help of arginine-rich peptides.[82],[83]

### SARS-COV-2 Thrives in Senescent Cells

Due to dysfunctional NKCs, macrophage/microglia, and resistance to apoptosis, senescent, virus-infected cells, may function as viral reservoirs, likely accounting for the protracted symptoms of long COVID.[84],[85] This is substantiated by the following findings:

(a) Multinucleated giant cells (syncytia) were found in SARS-COV-2 infected individuals as well as after vaccination with messenger RNA (mRNA) therapeutics.[86]-[88]

(b) Viral RNA has been detected in postmortem monocytes and macrophages derived from COVID-19 patients, indicates that senescent, virus-infected cells, resist phagocytic elimination and can thrive for a long interval of time.[89]

(c) The SARS-COV-2 virus usurps host NKCs via Nsp1 protein, compromising efferocytosis further and contributing to the accumulation of virus-infected, senescent cells.[90]

In contrast, SKF-38393, an allosteric modulator of sigma-1 receptors (Sig-1Rs) enhances macrophages/microglia-mediated efferocytosis of damaged cells, while protecting the healthy ones, including the neurons.[91], [92] Sig-1Rs also protect against SARS-COV-2 infection, likely by augmenting efferocytosis and the elimination of virus-infected infected cells. Furthermore candesartan, an inhibitor of intracellular Ca²⁺ influx, may block the activation of TMEM16F, decreasing cell-cell fusion and FIS.[94]

Taken together, the SARS-COV-2 virus induces premature cellular senescence and usurps the elimination of senescent virus-infected cells. This triggers inflammation and disruption of the gut barrier and BBB, allowing the translocation of gut microbes into host tissues and organs. Conversely, SKF38393 enhances the efferocytosis of senescent cells, while candesartan inhibits cell-cell fusion, together eliminating viral reservoirs and the symptoms of long COVID.

### Dysfunctional Efferocytosis and Long COVID

To maintain tissue homeostasis, millions of dead or dying cells need to be removed continuously by professional and non-professional phagocytes, including macrophages and NKCs. Overproduction of senescent cells or defective efferocytosis allows the survival of virus-infected cells, generating latent infection and long COVID.[95],[96] SARS-COV-2 binding to its receptor ACE-2, an ANG II degrading enzyme, contributes to ANG II accumulation. Upregulated ANG Il promotes the release of cortisol and HMGB1, molecules associated with fatigue, IBD, muscle weakness, and malignant transformation (FIG. 2).[97]-[99] In the gut, upregulation of cortisol and HMGB1 disrupts efferocytosis of intestinal epithelial cells (IECs) and ECs, contributing to microbial translocation (FIG. 2). As gut microbes are not immunologically tolerated outside the GI tract, they trigger inflammatory responses associated with fatiguing illnesses.[100] Moreover, ANG II is toxic for mitochondria, organelles previously associated with fatiguing illnesses.[101]-[103] Furthermore, ANG II increases the expression of intercellular adhesion molecule 1 (ICAM-1), a transmembrane glycoprotein, overexpressed in many pathological states, including frailty, depression, and cognitive impairment.[104]-[106] Dysfunctional ICAM-1 has been shown to disrupt efferocytosis, further contributing to the accumulation of senescent cells. Candesartan was demonstrated to inhibit the action of ANG II on ICAM-1, optimizing efferocytosis.[108]

### Stress and Biological Barriers

Under physiological conditions, the intestinal barrier allows absorption of nutrients, while preventing the migration of harmful substances, like toxins and bacteria, into the systemic circulation. Psychological stress and pathogens can disrupt this barrier, enabling translocation of microbes and/or their molecules into the body tissues and organs.[109],[110] The Hypothalamic-Pituitary-Adrenal (HPA) axis can be activated by both psychological and biological stressors, including viral infections, highlighting the link between stress, cellular senescence and barrier disruption.[111],[112]

Sterile inflammation refers to inflammasome activation by psychological and biological stressors which leads to numerous pathologies, including gut barrier dysfunction. [113],[114] It is well established that chronic stress triggers cortisol release via HPA, contributing to various diseases. Although cortisol exhibits robust anti-inflammatory properties, novel studies have reported that chronic cortisol release can activate Nod-like receptor protein 3 (NLRP3) inflammasome, inducing sterile inflammation that in turn disrupts the intestinal barrier. [115], For example, sterile inflammation in the GI tract has been associated with major depressive disorder (MDD) and Parkinson's disease, linking chronic stress and NLRP3 activation to neuropsychiatric pathology.[117],[118]

HMGB1, a damage-associated molecular pattern (DAMP) known for disrupting the gut barrier, is upregulated by both biological and psychological stress, highlighting a new target for microbial translocation disorders.[119]-[121] Like cortisol, HMGB1, was associated with premature cellular senescence, linking it to sterile inflammation.[122],[123]

Several studies have documented that D₁R active compounds can inhibit the NLRP3 inflammasome, lowering inflammatory responses, including sterile inflammation.[124] In addition, chronic psychological stress has been shown to lower brain dopamine levels, suggesting that D₁R agonists, including SKF38393 may reverse the detrimental effect of chronic stress. In this regard, cortisol-lowering properties of D₁R agonists has been utilized in the treatment of Cushing syndrome, suggesting that it can reverse cortisol and HNGB1-mediated sterile inflammation.[125], Moreover, candesartan was demonstrated to lower HMGB1, indicating that along with SKF38393, it could lower the effects of biological or psychological stressors on the intestinal barrier (130).[127]

### Dopamine Prevents Gray Matter Loss

COVID-19 infection has been associated with gray matter reduction, believed to take place via loss of dendritic arborization and spines.[128],[129] These structures form the bulk of the brain volume. In dementia, neurons die and are replaced by conjunctive tissue. In schizophrenia neurons remain alive but lose dendrites. Moreover, chronic pain, a key symptom of long COVID, has also been associated with brain thinning due to volume loss.[130] This is in line with novel voxel-based morphometry neuroimaging studies, suggesting that gray matter volume may be a reliable biological marker of long COVID.[131]

In normal aging, D₁R partial agonists/agonists can prevent and even restore the integrity of gray matter volume, restoring cognitive functions.[132] D₁R's are expressed perisynaptically (outside the synapse) and function similarly to autoreceptors. That is, DR partial agonists limit dopamine release when there is too much dopamine in the synapse, and promote dopamine release when there is insufficient dopamine in the synapse. Partial D₁R agonists, such as PF-06412562, like aripiprazole, are likely to restore both the brain volume and memory.[133] In this regard, the new class of non-catechol dopamine partial agonists (NCDPA, for example tavapadon), which can act as full agonists or partial agonists on the CAMP pathway, were demonstrated to restore working memory and the executive function in elderly with cognitive deficit.[134],[135]

Indeed, severe mental disorders and antipsychotic treatments were found to reduce the gray matter volume, suggesting the administration of a D₁R partial agonist along with neuroleptics may prevent gray matter depletion.[136],[137]

### Combinations

The administration of both a D₁R partial agonist and a BBB-crossing ARB provides a two-prong approach to treating long COVID, by first blocking entry of SARS-CoV-2 viral particles into cells that go on to become senescent and act as persistent viral reservoirs, and second by neutralizing already infected cells with a BBB-crossing ARB. Blocking D₁R increases dopamine in the synaptic cleft and inhibits mitochondrial trafficking.[138] The addition of an ARB is expected to augment this effect.

### Formulations

As discussed above, a D₁R partial agonists and an ARB are administered in either independently or in combination in this invention. Exemplary D₁R agonists and ARB's are disclosed above.

In an embodiment, a pharmaceutical formulation is provided including a D₁R agonist and an ARB in a unitary dosage form, i.e., a single dosage form containing a suitable dosage of both drugs. In an embodiment, the dosage form is a solid dosage form. In an alternative embodiment, a kit is provided in which a D₁R agonist and an ARB are provided in separate formulations and administered simultaneously or sequentially in time.

For the purposes of the invention, the term “solid dosage form” or “solid composition” refers to dosage forms such as tablets, capsules, or powders that are intended to be swallowed, i.e., used for oral administration. The solid compositions of the present invention can be prepared according to methods well known in the state of the art. Solid compositions typically are formulated with at least one excipient. The appropriate excipients and/or carriers, and their amounts, can readily be determined by those skilled in the art according to the type of formulation being prepared.

The pharmaceutical composition as defined above comprise appropriate excipients or carriers including, but not limited to, binders, fillers, disintegrants, glidants, lubricants or their mixtures. Additionally, the compositions of the present invention may contain other ingredients, such as flavoring agents and colorants, and other components known in the state of the art for use in pharmaceutical compositions.

The term “binder” refers to any pharmaceutically acceptable compound having binding properties. Materials commonly used as binders include povidone such as polyvinylpyrrolidone, methylcellulose polymers, hydroxyethyl cellulose, hydroxypropyl cellulose, L-hydroxypropyl cellulose (low substituted), hydroxypropylmethyl cellulose (HPMC), sodium carboxymethyl cellulose, carboxymethylene, carboxymethylhydroxyethyl cellulose and other cellulose derivatives, starches or modified starches, gelatine, sugars such as sucrose, glucose and sorbitol, gums such as sum arabic, tragacanth, agar and carragenenan; and mixture thereof. In an embodiment, the composition of the invention is one wherein the pharmaceutically or cosmetically acceptable excipients or carriers comprise one or more binder; preferably comprise polyvinylpyrrolidone. In an embodiment, the composition of the invention is one wherein the pharmaceutically or cosmetically acceptable excipients or carriers comprise one or more binder in an amount from 1% to 10% by weight, preferably from 1% to 6% by weight, more preferably from 1% to 3% by weight of the composition.

The terms “filler” and “diluent” have the same meaning and are used interchangeably. They refer to any pharmaceutically acceptable excipient or carrier (material) that fill out the size of a composition, making it practical to produce and convenient for the consumer to use. Materials commonly used as filler include calcium carbonate, calcium phosphate, dibasic calcium phosphate, tribasic calcium sulfate, calcium carboxymethyl cellulose, cellulose, cellulose products such as microcrystalline cellulose and its salts, dextrin derivatives, dextrin, dextrose, fructose, lactitol, lactose, starches or modified starches, magnesium carbonate, magnesium oxide, maltitol, maltodextrins, maltose, mannitol, sorbitol, starch, sucrose, sugar, xylitol, erythritol and mixtures thereof. In an embodiment, the composition of the invention is one wherein the pharmaceutically or cosmetically acceptable excipients or carriers comprises one or more filler; preferably comprises microcrystalline cellulose and its salts.

The term “disintegrant” refers to a substance which helps the composition break up once ingested. Materials commonly used as a disintegrant include cross linked polyvinylpyrolidone; starches such as maize starch and dried sodium starch glycolate; gums such as maize starch and dried sodium starch glycolate; gums such as alginic acid, sodium alginate, guar gum; croscarmellose sodium; low-substituted hydroxypropyl cellulose and mixtures thereof.

The term “glidant” refers to a substance which improves the flow characteristics of powder mixtures in the dry state. Materials commonly used as a glidant include magnesium stearate, colloidal silicon dioxide or talc. In an embodiment, the composition of the invention is one wherein the pharmaceutically or cosmetically acceptable excipients or carriers comprises one or more glidant; preferably comprises magnesium stearate, talc or mixture thereof.

The term “lubricant” refers to a substance that prevents composition ingredients from clumping together and from sticking to the tablet punches or capsule filling machine and improves flowability of the composition mixture. Materials commonly used as a lubricant include sodium oleate, sodium stearate, sodium benzoate, sodium stearate, sodium chloride, stearic acid, sodium stearyl fumarate, calcium stearate, magnesium stearate, magnesium lauryl sulfate, sodium stearyl fumarate, sucrose esters or fatty acid, zinc, polyethylene glycol, talc and mixtures thereof. The presence of a lubricant is particularly preferred when the composition is a tablet to improve the tableting process. In an embodiment, the composition of the invention is one wherein the pharmaceutically or cosmetically acceptable excipients or carriers comprises one or more lubricants; preferably comprises magnesium stearate.

The pharmaceutical compositions of the present invention can be prepared according to methods well known in the state of the art. The appropriate excipients and/or carriers, and their amounts, can readily be determined by those skilled in the art according to the type of formulation being prepared.

In an embodiment, the pharmaceutical composition is a tablet. In an embodiment, the pharmaceutical composition is a direct-compressed tablet. In an embodiment, the pharmaceutical composition is a capsule filled with a powder. In an embodiment, the pharmaceutical composition is a powder intended for suspension or dissolution in a beverage for oral consumption (a drinkable composition). In an embodiment, the dosage form may be a liquid for oral administration, such as a syrup.

Typically, oral dosage forms such as tablets and capsules are used with older children and competent adults who can take instruction and swallow a tablet or capsule. Liquid or drinkable dosage forms can be used with small children or incompetent adults who cannot swallow tablets or capsules.

### Dosages

Candesartan cilexetil and telmisartan are both approved drugs. Candesartan cilexetil may be administered in a single daily oral dose of 4-32 mg/day. Telmisartan is administered in a single daily oral dose of 40-80 mg/day.

No D₁R agonists are approved drugs. A published clinical trial for ecopipam used an oral dose 50-100 mg/day. [16] A clinical trial for SK38393 used 250 mg/day, characterized as a low dose, and failed to show a clinical benefit in schizophrenia. [139] A phase I clinical trial of PF-06649751 was published using dosages up to 15 mg/day for Parkinson's disease. The drug showed significant improvement of motor symptoms and was generally well tolerated in subjects with early-stage Parkinson's disease. [140]

In an embodiment, a unit dosage form for oral administration may be provided that is a single dosage form containing a D₁R agonist according to this invention, or an ARB according to this invention, or a combination thereof. Such a dosage form may be a tablet, capsule, liquid, or powder for dissolution or suspension in a liquid or beverage for oral consumption.

In an embodiment, a kit may be provided having separate oral dosage forms of a D₁R agonist and an ARB for administration to the patient. Such a kit can provide for a non-unit dose of the drug or for non-sequential administration of a combination of a D₁R agonist and an ARB.

### CONCLUSIONS

Long COVID is manifested by symptoms similar to those experienced by patients with fatiguing illnesses, including ME/CFS and CRF characterized by premature cellular senescence. The SARS-COV-2 virus is known for inducing early senescence, especially in epithelial, endothelial, and immune cells that comprise human biological barriers. In addition, senescent cells express an abundance of ACE-2 receptors which serve as viral entry portals, explaining the higher risk of COVID-19 critical illness for the elderly. Together this data suggests that the virus may utilize senescent cells as reservoirs, explaining the postmortem finding of viral proteins in monocytes and macrophages derived from COVID-19 deceased patients. Like HIV, SARS-COV-2 thriving in microglia may account for the neuropsychiatric sequelae experienced by many patients with long COVID. D₁R agonists and BBB-crossing ARB's such as candesartan exert anti-inflammatory, pro-cognitive, and senolytic properties, indicating likely efficacy in clearing viral reservoirs.

### GLOSSARY

ACE-2 angiotensin converting enzyme 2

ANG II angiotensin II

ARB angiotensin receptor blocker

AT₁R angiotensin II type 1 receptor

BBB Blood-brain barrier

CaMKII Ca²⁺/calmodulin-dependent protein kinase II

CRF Cancer-related fatigue

D₁R dopamine receptor 1

DAMP Damage-associated molecular pattern

EC endothelial cell

FIS fusion-induced senescence

GPCR G protein-coupled receptors

HERV human endogenous retrovirus

HMGB1 High-mobility group box 1

HPA Hypothalamic-Pituitary-Adrenal axis

ICAM-1 Intercellular adhesion molecule 1

IEC Intestinal epithelial cells

LPS Lipopolysaccharide

ME/CFS Myalgic Encephalomyelitis/Chronic Fatigue Syndrome

NKC Natural killer cells

NLRP3 Nod-like receptor protein 3

SASP Senescence-associated secretory phenotype

S protein SARS-COV-2 spike protein

Sig-1R Sigma-1 receptor

TMEM16F Ca²⁺ dependent phospholipid scramblase, see ref. [81]

VIS Virus induced senescence

### REFERENCES

(1) Huang, W.; Hickson, L. J.; et al. Cellular Senescence: The Good, the Bad and the Unknown. Nat Rev Nephrol 2022, 18 (10), 611-627. https://doi.org/10.1038/s41581-022-00601-z.

(2) Dalzini, A.; Ballin, G.; et al. Size of HIV-1 Reservoir Is Associated with Telomere Shortening and Immunosenescence in Early-treated European Children with Perinatally Acquired HIV-1. J Int AIDS Soc 2021, 24 (11). https://doi.org/10.1002/jia2.25847.

(3) Sepe, S.; Rossiello, F.; et al. DNA Damage Response at Telomeres Boosts the Transcription of SARS-COV-2 Receptor ACE2 during Aging. EMBO Rep 2022, 23(2). https://doi.org/10.15252/embr.202153658.

(4) Osorio, C.; Sfera, A.; et al. Virus-Induced Membrane Fusion in Neurodegenerative Disorders. Frontiers in Cellular and Infection Microbiology. Frontiers Media S.A. Mar. 24, 2022. https://doi.org/10.3389/fcimb.2022.845580.

(5) Osorio, C.; Kozlakidis, Z.; et al. Efferocytosis and Biological Barriers Subjects: Cell Biology; 2023.

(6) Meng, L.; Wang, M.; et al. Dopamine D1 Receptor Agonist Alleviates Acute Lung Injury via Modulating Inflammatory Responses in Macrophages and Barrier Function in Airway Epithelial Cells. Free Radic Biol Med 2023, 202, 2-16. https://doi.org/10.1016/j.freeradbiomed.2023.03.016.

(7) Qie, S.; Ran, Y.; et al. Candesartan Modulates Microglia Activation and Polarization via NF-KB Signaling Pathway. Int J Immunopathol Pharmacol 2020, 34, 205873842097490. https://doi.org/10.1177/2058738420974900.

(8) Elkahloun, A. G.; Saavedra, J. M. Candesartan Neuroprotection in Rat Primary Neurons Negatively Correlates with Aging and Senescence: A Transcriptomic Analysis. Mol Neurobiol 2020, 57 (3), 1656-1673. https://doi.org/10.1007/s12035-019-01800-9.

(9) Birge, R. B.; Boeltz, S.; et al. Phosphatidylserine Is a Global Immunosuppressive Signal in Efferocytosis, Infectious Disease, and Cancer. Cell Death Differ 2016, 23 (6), 962-978. https://doi.org/10.1038/cdd.2016.11.

(10) Nickoloff-Bybel, E. A.; Mackie, P.; et al. Dopamine Increases HIV Entry into Macrophages by Increasing Calcium Release via an Alternative Signaling Pathway. Brain Behav Immun 2019, 82, 239-252.https://doi.org/10.1016/j.bbi.2019.08.191.

(11) Khalefah, M. M.; Khalifah, A. M. Determining the Relationship between SARS-CoV-2 Infection, Dopamine, and COVID-19 Complications. J Taibah Univ Med Sci 2020, 15 (6), 550-553. https://doi.org/10.1016/j.jtumed.2020.10.006.

(12) Yang, L.; Kim, T. W.; et al. SARS-COV-2 Infection Causes Dopaminergic Neuron Senescence. Cell Stem Cell 2024, 31 (2), 196-211.e6. https://doi.org/10.1016/j.stem.2023.12.012.

(13) Tan, L.; Yan, W.; et al. Biased Ligands of G Protein-Coupled Receptors (GPCRs): Structure-Functional Selectivity Relationships (SFSRs) and Therapeutic Potential. J Med Chem 2018, 61 (22), 9841-9878. https://doi.org/10.1021/acs.jmedchem.8b00435.

(14) Gray, D. L.; Allen, J. A.; et al. Impaired β-Arrestin Recruitment and Reduced Desensitization by Non-Catechol Agonists of the D1 Dopamine Receptor. Nat Commun 2018, 9 (1), 674. https://doi.org/10.1038/s41467-017-02776-7.

(15) Kee, T. R.; Khan, S. A.; et al. The Multifaceted Functions of β-Arrestins and Their Therapeutic Potential in Neurodegenerative Diseases. Exp Mol Med 2024, 56 (1), 129-141. https://doi.org/10.1038/s12276-023-01144-4.

(16) Gilbert, D. L.; Dubow, J. S.; et al. Ecopipam for Tourette Syndrome: A Randomized Trial. Pediatrics 2023, 151 (2). https://doi.org/10.1542/peds.2022-059574.

(17) Kocienski, P. Synthesis of Ecopipam. Synfacts 2012, 8 (07), 0704-0704.https://doi.org/10.1055/s-0031-1290421.

(18) Jones-Tabah, J.; Mohammad, H.; et al. The Signaling and Pharmacology of the Dopamine D1 Receptor. Front Cell Neurosci 2022, 15. https://doi.org/10.3389/fncel.2021.806618.

(19) Nutt, D.; Lingford-Hughes, A. Addiction: The Clinical Interface. Br J Pharmacol 2008, 154 (2), 397-405. https://doi.org/10.1038/bjp.2008.101.

(20) Lawrence, A. J.; Beart, P. M.; et al. Neuropharmacology of Addiction—Setting the Scene. Br J Pharmacol 2008, 154 (2), 259-260. https://doi.org/10.1038/bjp.2008.131.

(21) Arnsten, A. F. T.; Cai, J. X.; et al. Dopamine D1 Receptor Mechanisms in the Cognitive Performance of Young Adult and Aged Monkeys. Psychopharmacology(Berl) 1994, 116 (2), 143-151. https://doi.org/10.1007/BF02245056.

(22) Hall, A.; Provins, L.; et al. Novel Strategies To Activate the Dopamine D1Receptor: Recent Advances in Orthosteric Agonism and Positive Allosteric Modulation. J Med Chem 2018, 62 (1), 128-140. https://doi.org/10.1021/acs.jmedchem.8b01767.

(23) Samudyata; Oliveira, A. O.; et al. SARS-COV-2 Promotes Microglial Synapse Elimination in Human Brain Organoids. Mol Psychiatry 2022, 27 (10), 3939-3950.https://doi.org/10.1038/s41380-022-01786-2.

(24) Ho, J. K.; Moriarty, F.; et al. Blood-Brain Barrier Crossing Renin-Angiotensin Drugs and Cognition in the Elderly: A Meta-Analysis. Hypertension 2021, 78 (3), 629-643. https://doi.org/10.1161/HYPERTENSIONAHA.121.17049.

(25) Lynch, S. M.; Guo, G.; et al. Role of Senescence and Aging in SARS-COV-2Infection and COVID-19 Disease. Cells 2021, 10 (12), 3367. https://doi.org/10.3390/cells10123367.

(26) Suzuki, K.; Susaki, E. A.; et al. Lipopolysaccharides and Cellular Senescence: Involvement in Atherosclerosis. Int J Mol Sci 2022, 23 (19), 11148.https://doi.org/10.3390/ijms231911148.

(27) Schmitt, C. A.; Tchkonia, T.; et al. COVID-19 and Cellular Senescence. Nat Rev Immunol 2023, 23 (4), 251-263. https://doi.org/10.1038/s41577-022-00785-2.

(28) Lee, S.; Yu, Y.; et al. Virus-Induced Senescence Is a Driver and Therapeutic Target in COVID-19. Nature 2021, 599 (7884), 283-289. https://doi.org/10.1038/s41586-021-03995-1.

(29) Sfera, A.; Osorio, C.; et al. COVID-19, Ferrosenescence and Neurodegeneration, a Mini-Review. Prog Neuropsychopharmacol Biol Psychiatry 2021, 109, 110230. https://doi.org/10.1016/j.pnpbp.2020.110230.

(30) Osorio, C.; Sfera, A.; et al. Virus-Induced Membrane Fusion in Neurodegenerative Disorders. Front Cell Infect Microbiol 2022, 12. https://doi.org/10.3389/fcimb.2022.845580.

(31) Gal, H.; Krizhanovsky, V. Cell Fusion Induced Senescence. Aging 2014, 6 (5), 353-354. https://doi.org/10.18632/aging.100670.

(32) Chuprin, A.; Gal, H.; et al. Cell Fusion Induced by ERVWE1 or Measles Virus Causes Cellular Senescence. Genes Dev 2013, 27 (21), 2356-2366.https://doi.org/10.1101/gad.227512.113.

(33) Burton, D. G. A.; Krizhanovsky, V. Physiological and Pathological Consequences of Cellular Senescence. Cellular and Molecular Life Sciences 2014, 71 (22), 4373-4386. https://doi.org/10.1007/s00018-014-1691-3.

(34) Sfera, A.; Osorio, C.; et al. Endothelial Senescence and Chronic Fatigue Syndrome, a COVID-19 Based Hypothesis. Front Cell Neurosci 2021, 15.https://doi.org/10.3389/fncel.2021.673217.

(35) Sfera, A.; Osorio, C.; et al. Long COVID and the Neuroendocrinology of Microbial Translocation Outside the GI Tract: Some Treatment Strategies. Endocrines 2022, 3 (4), 703-725. https://doi.org/10.3390/endocrines3040058.

(36) Varesi, A.; Deumer, U.-S.; et al. The Emerging Role of Gut Microbiota in Myalgic Encephalomyelitis/Chronic Fatigue Syndrome (ME/CFS): Current Evidence and Potential Therapeutic Applications. J Clin Med 2021, 10 (21), 5077.https://doi.org/10.3390/jcm10215077.

(37) Servaes, P.; Gielissen, M. F. M.; et al. The Course of Severe Fatigue in Disease-free Breast Cancer Patients: A Longitudinal Study. Psychooncology 2007, 16 (9), 787-795. https://doi.org/10.1002/pon.1120.

(38) Savina, S.; Zaydiner, B. Cancer-Related Fatigue: Some Clinical Aspects. Asia Pac J Oncol Nurs 2019, 6 (1), 7-9. https://doi.org/10.4103/apjon.apjon_45_18.

(39) Thong, M. S. Y.; van Noorden, C. J. F.; et al. Cancer-Related Fatigue: Causes and Current Treatment Options. Curr Treat Options Oncol 2020, 21 (2), 17.https://doi.org/10.1007/s11864-020-0707-5.

(40) Zhang, J.-W.; Zhang, D.; et al. Senescent Cells in Cancer Therapy: Why and How to Remove Them. Cancer Lett 2021, 520, 68-79.https://doi.org/10.1016/j.canlet.2021.07.002.

(41) Unger, T. Pharmacology of AT 1-Receptor Blockers. Blood Press 2001, 10 (3), 5-10. https://doi.org/10.1080/08037050152518302.

(42) Fan, F.; Tian, C.; et al. Candesartan Attenuates Angiogenesis in Hepatocellular Carcinoma via Downregulating AT1R/VEGF Pathway. Biomedicine & Pharmacotherapy 2016, 83, 704-711.https://doi.org/10.1016/j.biopha.2016.07.039.

(43) Johnson, D. E.; Ochieng, J.; et al. The Growth Inhibitory Properties of a Dopamine Agonist (SKF 38393) on MCF-7 Cells. Anticancer Drugs 1995, 6 (3), 471-474. https://doi.org/10.1097/00001813-199506000-00017.

(44) Sobczuk, P.; Łomiak, M.; et al. Dopamine D1 Receptor in Cancer. Cancers (Basel) 2020, 12 (11), 3232. https://doi.org/10.3390/cancers12113232.

(45) Dobryakova, E.; Genova, H. M.; et al. The Dopamine Imbalance Hypothesis of Fatigue in Multiple Sclerosis and Other Neurological Disorders. Front Neurol 2015, 6. https://doi.org/10.3389/fneur.2015.00052.

(46) McCaffrey, D.; Lawther, A. J.; et al. Cancer Activates Microglia to the Same Extent as Chronic Stress throughout Stress Neurocircuitry in a Mouse Model of Breast Cancer. Psychoneuroendocrinology 2022, 146, 105938.https://doi.org/10.1016/j.psyneuen.2022.105938.

(47) Kavanagh, E. Long Covid Brain Fog: A Neuroinflammation Phenomenon? Oxf Open Immunol 2022, 3 (1). https://doi.org/10.1093/oxfimm/iqac007.

(48) Mavoungou, E.; Bouyou-Akotet, M. K.; et al. Effects of Prolactin and Cortisol on Natural Killer (NK) Cell Surface Expression and Function of Human Natural Cytotoxicity Receptors (NKp46, NKp44 and NKp30). Clin Exp Immunol 2004, 139(2), 287-296. https://doi.org/10.1111/j.1365-2249.2004.02686.x.

(49) Meeus, M.; Mistiaen, W.; et al. Immunological Similarities between Cancer and Chronic Fatigue Syndrome: The Common Link to Fatigue? Anticancer Res 2009, 29 (11), 4717-4726.

(50) Wu, Y.; Luo, X.; et al. The Disbalance of LRP1 and SIRPα by Psychological Stress Dampens the Clearance of Tumor Cells by Macrophages. Acta Pharm Sin B 2022, 12 (1), 197-209. https://doi.org/10.1016/j.apsb.2021.06.002.

(51) Wang, Y.; Zhang, W.; et al. Extracellular HMGB1 Impairs Macrophage-Mediated Efferocytosis by Suppressing the Rab43-Controlled Cell Surface Transport of CD91. Front Immuno/2022, 13. https://doi.org/10.3389/fimmu.2022.767630.

(52) Martin-Rodriguez, O.; Gauthier, T.; et al. Pro-Resolving Factors Released by Macrophages After Efferocytosis Promote Mucosal Wound Healing in Inflammatory Bowel Disease. Front Immuno/2021, 12. https://doi.org/10.3389/fimmu.2021.754475.

(53) Krouwer, V. J. D.; Hekking, L. H. P.; et al. Endothelial Cell Senescence Is Associated with Disrupted Cell-Cell Junctions and Increased Monolayer Permeability. Vasc Cell 2012, 4 (1), 12. https://doi.org/10.1186/2045-824X-4-12.

(54) Yamazaki, Y.; Baker, D. J.; et al. Vascular Cell Senescence Contributes to Blood-Brain Barrier Breakdown. Stroke 2016, 47 (4), 1068-1077.https://doi.org/10.1161/STROKEAHA.115.010835.

(55) Jurewicz, M.; McDermott, D. H.; et al. Human T and Natural Killer Cells Possess a Functional Renin-Angiotensin System. Journal of the American Society of Nephrology 2007, 18 (4), 1093-1102. https://doi.org/10.1681/ASN.2006070707.

(56) Meng, L.; Wang, M.; et al. Dopamine D1 Receptor Agonist Alleviates Acute Lung Injury via Modulating Inflammatory Responses in Macrophages and Barrier Function in Airway Epithelial Cells. Free Radic Biol Med 2023, 202, 2-16.https://doi.org/10.1016/j.freeradbiomed.2023.03.016.

(57) Zhao, W.; Huang, Y.; et al. Dopamine Receptors Modulate Cytotoxicity of Natural Killer Cells via CAMP-PKA-CREB Signaling Pathway. PLOS One 2013, 8 (6), e65860. https://doi.org/10.1371/journal.pone.0065860.

(58) Qie, S.; Ran, Y.; et al. Candesartan Modulates Microglia Activation and Polarization via NF-KB Signaling Pathway. Int J Immunopathol Pharmacol 2020, 34, 205873842097490. https://doi.org/10.1177/2058738420974900.

(59) Wallet, C.; De Rovere, M.; et al. Microglial Cells: The Main HIV-1 Reservoir in the Brain. Front Cell Infect Microbiol 2019, 9. https://doi.org/10.3389/fcimb.2019.00362.

(60) Masaldan, S.; Clatworthy, S. A. S.; et al. Iron Accumulation in Senescent Cells Is Coupled with Impaired Ferritinophagy and Inhibition of Ferroptosis. Redox Biol 2018, 14, 100-115. https://doi.org/10.1016/j.redox.2017.08.015.

(61) Pence, B.; Zhang, Y.; et al. Senescent Macrophages Alter Fibroblast Fibrogenesis in Response to SARS-COV-2 Infection. Neurolmmune Pharmacology and Therapeutics 2022, 1 (1), 37-42. https://doi.org/10.1515/nipt-2022-0003.

(62) Hama Amin, B. J.; Kakamad, F. H.; et al. Post COVID-19 Pulmonary Fibrosis; a Meta-Analysis Study. Annals of Medicine & Surgery 2022, 77.https://doi.org/10.1016/j.amsu.2022.103590.

(63) Samudyata; Oliveira, A. O.; et al. SARS-COV-2 Promotes Microglial Synapse Elimination in Human Brain Organoids. Mol Psychiatry 2022, 27 (10), 3939-3950.https://doi.org/10.1038/s41380-022-01786-2.

(64) Sato, T.; Shapiro, J. S.; et al. Aging Is Associated with Increased Brain Iron through Cortex-Derived Hepcidin Expression. Elife 2022, 11.https://doi.org/10.7554/eLife.73456.

(65) Di Micco, R.; Krizhanovsky, V.; et al. Cellular Senescence in Ageing: From Mechanisms to Therapeutic Opportunities. Nat Rev Mol Cell Biol 2021, 22 (2), 75-95. https://doi.org/10.1038/s41580-020-00314-w.

(66) Martin, N.; Bernard, D. Calcium Signaling and Cellular Senescence. Cell Calcium 2018, 70, 16-23. https://doi.org/10.1016/j.ceca.2017.04.001.

(67) Schmitt, C. A.; Tchkonia, T.; et al. COVID-19 and Cellular Senescence. Nat Rev Immunol 2023, 23 (4), 251-263. https://doi.org/10.1038/s41577-022-00785-2.

(68) Gioia, U.; Tavella, S.; et al. SARS-COV-2 Infection Induces DNA Damage, through CHK1 Degradation and Impaired 53BP1 Recruitment, and Cellular Senescence. Nat Cell Biol 2023, 25 (4), 550-564. https://doi.org/10.1038/s41556-023-01096-x.

(69) Yang, L.; Kim, T. W.; et al. SARS-COV-2 Infection Causes Dopaminergic Neuron Senescence. Cell Stem Cell 2024, 31 (2), 196-211.e6.https://doi.org/10.1016/j.stem.2023.12.012.

(70) Sepe, S.; Rossiello, F.; et al. DNA Damage Response at Telomeres Boosts the Transcription of SARS-COV-2 Receptor ACE2 during Aging. i EMBO Rep 2022, 23(2). https://doi.org/10.15252/embr.202153658.

(71) Zhao, W.; Huang, Y.; et al. Dopamine Receptors Modulate Cytotoxicity of Natural Killer Cells via CAMP-PKA-CREB Signaling Pathway. PLOS One 2013, 8 (6), e65860. https://doi.org/10.1371/journal.pone.0065860.

(72) Mirzayans, R.; Andrais, B.; et al. Multinucleated Giant Cancer Cells Produced in Response to lonizing Radiation Retain Viability and Replicate Their Genome. Int J Mol Sci 2017, 18 (2), 360. https://doi.org/10.3390/ijms18020360.

(73) Blaise, S.; de Parseval, N.; et al. Genomewide Screening for Fusogenic Human Endogenous Retrovirus Envelopes Identifies Syncytin 2, a Gene Conserved on Primate Evolution. Proceedings of the National Academy of Sciences 2003, 100(22), 13013-13018. https://doi.org/10.1073/pnas.2132646100.

(74) Dittmar, T.; Weiler, J.; et al. Cell-Cell Fusion Mediated by Viruses and HERV-Derived Fusogens in Cancer Initiation and Progression. Cancers (Basel) 2021, 13(21), 5363. https://doi.org/10.3390/cancers13215363.

(75) Nelson, P. N. Demystified . . . Human Endogenous Retroviruses. Molecular Pathology 2003, 56 (1), 11-18. https://doi.org/10.1136/mp.56.1.11.

(76) Wang, X.; Wu, X.; et al. Human Endogenous Retrovirus W Family Envelope Protein (HERV-W Env) Facilitates the Production of TNF-α and IL-10 by Inhibiting MyD88s in Glial Cells. Arch Virol 2021, 166 (4), 1035-1045.https://doi.org/10.1007/s00705-020-04933-8.

(77) Denner, J. Expression and Function of Endogenous Retroviruses in the Placenta. APMIS 2016, 124 (1-2), 31-43. https://doi.org/10.1111/apm.12474.

(78) Giménez-Orenga, K.; Pierquin, J.; et al. HERV-W ENV Antigenemia and Correlation of Increased Anti-SARS-COV-2 Immunoglobulin Levels with Post-COVID-19 Symptoms. Front Immunol 2022, 13.https://doi.org/10.3389/fimmu.2022.1020064.

(79) Rodrigues, L. S.; da Silva Nali, L. H.; et al. HERV-K and HERV-W Transcriptional Activity in Myalgic Encephalomyelitis/Chronic Fatigue Syndrome. Autoimmunity Highlights 2019, 10 (1), 12. https://doi.org/10.1186/s13317-019-0122-8.

(80) Zhang, M.; Liang, J. Q.; et al. Expressional Activation and Functional Roles of Human Endogenous Retroviruses in Cancers. Rev Med Virol 2019, 29 (2). https://doi.org/10.1002/rmv.2025.

(81) Whitlock, J. M.; Chernomordik, L. V. Flagging Fusion: Phosphatidylserine Signaling in Cell-Cell Fusion. Journal of Biological Chemistry 2021, 296, 100411.https://doi.org/10.1016/j.jbc.2021.100411.

(82) Wenzhong, L.; Hualan, L. COVID-19: The CaMKII-like System of S Protein Drives Membrane Fusion and Induces Syncytial Multinucleated Giant Cells. Immunol Res 2021, 69 (6), 496-519. https://doi.org/10.1007/s12026-021-09224-1.

(83) Rong, Z.; Mai, H.; et al. SARS-COV-2 Spike Protein Accumulation in the Skull-Meninges-Brain Axis: Potential Implications for Long-Term Neurological Complications in Post-COVID-19. bioRxiv 2023, 2023.04.04.535604.https://doi.org/10.1101/2023.04.04.535604.

(84) Miron, R. J.; Bosshardt, D. D. Multinucleated Giant Cells: Good Guys or Bad Guys? Tissue Eng Part B Rev 2018, 24 (1), 53-65.https://doi.org/10.1089/ten.teb.2017.0242.

(85) Rong, Z.; Mai, H.; et al. SARS-COV-2 Spike Protein Accumulation in the Skull-Meninges-Brain Axis: Potential Implications for Long-Term Neurological Complications in Post-COVID-19. bioRxiv 2023, 2023.04.04.535604.https://doi.org/10.1101/2023.04.04.535604.

(86) Stadlmann, S.; Hein-Kuhnt, R.; et al. Viropathic Multinuclear Syncytial Giant Cells in Bronchial Fluid from a Patient with COVID-19. J Clin Pathol 2020, 73 (9), 607-608. https://doi.org/10.1136/jclinpath-2020-206657.

(87) Sung, K.; McCain, J.; et al. Biopsy-Proven Giant Cell Myocarditis Following the COVID-19 Vaccine. Circ Heart Fail 2022, 15 (4). https://doi.org/10.1161/CIRCHEARTFAILURE.121.009321.

(88) Buchrieser, J.; Dufloo, J.; et al. Syncytia Formation by SARS-COV-2-infected Cells. EMBO J 2020, 39 (23). https://doi.org/10.15252/embj.2020106267.

(89) Jalloh, S.; Olejnik, J.; et al. CD169-Mediated Restrictive SARS-COV-2 Infection of Macrophages Induces pro-Inflammatory Responses. PLOS Pathog 2022, 18 (10), e1010479. https://doi.org/10.1371/journal.ppat.1010479.

(90) Lee, M. J.; Leong, M. W.; et al. SARS-COV-2 Escapes Direct NK Cell Killing through Nsp1-Mediated Downregulation of Ligands for NKG2D. Cell Rep 2022, 41(13), 111892. https://doi.org/10.1016/j.celrep.2022.111892.

(91) Guo, L.; Chen, Y.; et al. Allosteric Modulation of Sigma-1 Receptors Elicits Anti-seizure Activities. Br J Pharmacol 2015, 172 (16), 4052-4065.https://doi.org/10.1111/bph.13195.

(92) Zhang, G.; Li, Q.; et al. Sigma-1 Receptor-Regulated Efferocytosis by Infiltrating Circulating Macrophages/Microglial Cells Protects against Neuronal Impairments and Promotes Functional Recovery in Cerebral Ischemic Stroke. Theranostics 2023, 13 (2), 543-559. https://doi.org/10.7150/thno.77088.

(93) Vela, J. M. Repurposing Sigma-1 Receptor Ligands for COVID-19 Therapy? Front Pharmacol 2020, 11. https://doi.org/10.3389/fphar.2020.582310.

(94) Chang, G.; Yeh, Y.; et al. Candesartan Cilexetil Attenuates Arrhythmogenicity Following Pressure Overload in Rats via the Modulation of Cardiac Electrical and Structural Remodeling and Calcium Handling Dysfunction. J Am Heart Assoc 2022, 11 (15). https://doi.org/10.1161/JAHA.121.024285.

(95) Gerlach, B. D.; Ampomah, P. B.; et al. Efferocytosis Induces Macrophage Proliferation to Help Resolve Tissue Injury. Cell Metab 2021, 33 (12), 2445-2463.e8. https://doi.org/10.1016/j.cmet.2021.10.015.

(96) Kawano, M.; Nagata, S. Efferocytosis and Autoimmune Disease. Int Immunol 2018, 30 (12), 551-558. https://doi.org/10.1093/intimm/dxy055.

(97) Kvivik, I.; Grimstad, T.; et al. Anti-HMGB1 Auto-Abs Influence Fatigue in Patients with Crohn's Disease. Innate Immun 2021, 27 (4), 286-293.https://doi.org/10.1177/17534259211014252.

(98) Ho, T.-L.; Tang, C.-H.; et al. HMGB1 Promotes In Vitro and In Vivo Skeletal Muscle Atrophy through an IL-18-Dependent Mechanism. Cells 2022, 11 (23), 3936. https://doi.org/10.3390/cells11233936.

(99) Chen, R.; Zou, J.; et al. The Redox Protein HMGB1 in Cell Death and Cancer. Antioxid Redox Signal 2023. https://doi.org/10.1089/ars.2023.0007.

(100) Komaroff, A. L. Inflammation Correlates with Symptoms in Chronic Fatigue Syndrome. Proceedings of the National Academy of Sciences 2017, 114 (34), 8914-8916. https://doi.org/10.1073/pnas.1712475114.

(101) Morris, G.; Maes, M. Mitochondrial Dysfunctions in Myalgic Encephalomyelitis/Chronic Fatigue Syndrome Explained by Activated Immuno-Inflammatory, Oxidative and Nitrosative Stress Pathways. Metab Brain Dis 2014, 29 (1), 19-36.https://doi.org/10.1007/s11011-013-9435-x.

(102) Silva, K. A. S.; Ghiarone, T.; et al. Angiotensin II Suppresses Autophagy and Disrupts Ultrastructural Morphology and Function of Mitochondria in Mouse Skeletal Muscle. J Appl Physiol 2019, 126 (6), 1550-1562.https://doi.org/10.1152/japplphysiol.00898.2018.

(103) Refolo, G.; Vescovo, T.; et al. Mitochondrial Interactome: A Focus on Antiviral Signaling Pathways. Front Cell Dev Biol 2020, 8.https://doi.org/10.3389/fcell.2020.00008.

(104) Baghai, T.; Varallo-Bedarida, G.; et al. Classical Risk Factors and Inflammatory Biomarkers: One of the Missing Biological Links between Cardiovascular Disease and Major Depressive Disorder. Int J Mol Sci 2018, 19 (6), 1740.https://doi.org/10.3390/ijms19061740.

(105) Nielsen, H. M.; Londos, E.; et al. Soluble Adhesion Molecules and Angiotensin-Converting Enzyme in Dementia. Neurobiol Dis 2007, 26 (1), 27-35.https://doi.org/10.1016/j.nbd.2006.11.011.

(106) Lee, W.-J.; Chen, L.-K.; et al. Soluble ICAM-1, Independent of IL-6, Is Associated with Prevalent Frailty in Community-Dwelling Elderly Taiwanese People. PLOS One 2016, 11 (6), e0157877. https://doi.org/10.1371/journal.pone.0157877.

(107) Wiesolek, H. L.; Bui, T. M.; et al. Intercellular Adhesion Molecule 1 Functions as an Efferocytosis Receptor in Inflammatory Macrophages. Am J Pathol 2020, 190(4), 874-885. https://doi.org/10.1016/j.ajpath.2019.12.006.

(108) Pastore, L.; Tessitore, A.; et al. Angiotensin II Stimulates Intercellular Adhesion Molecule-1 (ICAM-1) Expression by Human Vascular Endothelial Cells and Increases Soluble ICAM-1 Release In Vivo. Circulation 1999, 100 (15), 1646-1652. https://doi.org/10.1161/01.CIR.100.15.1646.

(109) Varanoske, A. N.; McClung, H. L.; et al. Stress and the Gut-Brain Axis: Cognitive Performance, Mood State, and Biomarkers of Blood-Brain Barrier and Intestinal Permeability Following Severe Physical and Psychological Stress. Brain Behav Immun 2022, 101, 383-393. https://doi.org/10.1016/j.bbi.2022.02.002.

(110) Mawdsley, J. E. Psychological Stress in IBD: New Insights into Pathogenic and Therapeutic Implications. Gut 2005, 54 (10), 1481-1491.https://doi.org/10.1136/gut.2005.064261.

(111) Silverman, M. N.; Pearce, B. D.; et al. Immune Modulation of the Hypothalamic-Pituitary-Adrenal (HPA) Axis during Viral Infection. Viral Immunol 2005, 18 (1), 41-78. https://doi.org/10.1089/vim.2005.18.41.

(112) Chrousos, G. P.; Zapanti, E. D. Hypothalamic-Pituitary-Adrenal Axis in HIV Infection and Disease. Endocrinol Metab Clin North Am 2014, 43 (3), 791-806.https://doi.org/10.1016/j.ecl.2014.06.002.

(113) Iwata, M.; Ota, K. T.; et al. The Inflammasome: Pathways Linking Psychological Stress, Depression, and Systemic Illnesses. Brain Behav Immun 2013, 31, 105-114. https://doi.org/10.1016/j.bbi.2012.12.008.

(114) Fleshner, M.; Frank, M.; et al. Danger Signals and Inflammasomes: Stress-Evoked Sterile Inflammation in Mood Disorders. Neuropsychopharmacology 2017, 42 (1), 36-45. https://doi.org/10.1038/npp.2016.125.

(115) Feng, X.; Zhao, Y.; et al. Glucocorticoid-Driven NLRP3 Inflammasome Activation in Hippocampal Microglia Mediates Chronic Stress-Induced Depressive-Like Behaviors. Front Mol Neurosci 2019, 12.https://doi.org/10.3389/fnmol.2019.00210.

(116) Bharti, V.; Tan, H.; et al. Txnip Mediates Glucocorticoid-Activated NLRP3Inflammatory Signaling in Mouse Microglia. Neurochem Int 2019, 131, 104564.https://doi.org/10.1016/j.neuint.2019.104564.

(117) Shannon, K. M. Gut-Derived Sterile Inflammation and Parkinson's Disease. Front Neurol 2022, 13. https://doi.org/10.3389/fneur.2022.831090.

(118) Franklin, T. C.; Xu, C.; et al. Depression and Sterile Inflammation: Essential Role of Danger Associated Molecular Patterns. Brain Behav Immun 2018, 72, 2-13.https://doi.org/10.1016/j.bbi.2017.10.025.

(119) Weber, M. D.; Frank, M. G.; et al. Stress Induces the Danger-Associated Molecular Pattern HMGB-1 in the Hippocampus of Male Sprague Dawley Rats: A Priming Stimulus of Microglia and the NLRP3 Inflammasome. The Journal of Neuroscience 2015, 35 (1), 316-324. https://doi.org/10.1523/JNEUROSCI.3561-14.2015.

(120) Wu, T.-Y.; Liu, L.; et al. High-Mobility Group Box-1 Was Released Actively and Involved in LPS Induced Depressive-like Behavior. J Psychiatr Res 2015, 64, 99-106. https://doi.org/10.1016/j.jpsychires.2015.02.016.

(121) Chen, X.; Zhao, H.-X.; et al. Blockade of High-Mobility Group Box 1 Attenuates Intestinal Mucosal Barrier Dysfunction in Experimental Acute Pancreatitis. Sci Rep 2017, 7 (1), 6799. https://doi.org/10.1038/s41598-017-07094-y.

(122) Gao, X.; Li, F.; et al. Cellular Senescence in Adrenocortical Biology and Its Disorders. Cells 2021, 10 (12), 3474. https://doi.org/10.3390/cells10123474.

(123) Lee, J.-J.; Park, I. H.; et al. HMGB1 Orchestrates STING-Mediated Senescence via TRIM30α Modulation in Cancer Cells. Cell Death Discov 2021, 7 (1), 28.https://doi.org/10.1038/s41420-021-00409-z.

(124) Jiang, W.; Li, M.; et al. Dopamine D1 Receptor Agonist A-68930 Inhibits NLRP3Inflammasome Activation and Protects Rats from Spinal Cord Injury-Induced Acute Lung Injury. Spinal Cord 2016, 54 (11), 951-956.https://doi.org/10.1038/sc.2016.52.

(125) Petrossians, P.; Thonnard, A.-S.; et al. Medical Treatment in Cushing's Syndrome: Dopamine Agonists and Cabergoline. Neuroendocrinology 2010, 92(Suppl. 1), 116-119. https://doi.org/10.1159/000317716.

(126) Bloomfield, M. A.; Mccutcheon, R. A.; et al. The Effects of Psychosocial Stress on Dopaminergic Function and the Acute Stress Response. Elife 2019, 8.https://doi.org/10.7554/eLife.46797.

(127) Kikuchi, K.; Tancharoen, S.; et al. Potential of the Angiotensin Receptor Blockers (ARBs) Telmisartan, Irbesartan, and Candesartan for Inhibiting the HMGB1/RAGE Axis in Prevention and Acute Treatment of Stroke. Int J Mol Sci 2013, 14 (9), 18899-18924. https://doi.org/10.3390/ijms140918899.

(128) Douaud, G.; Lee, S.; et al. SARS-COV-2 Is Associated with Changes in Brain Structure in UK Biobank. Nature 2022, 604 (7907), 697-707.https://doi.org/10.1038/s41586-022-04569-5.

(129) Jan, Y.-N.; Jan, L. Y. Branching out: Mechanisms of Dendritic Arborization. Nat Rev Neurosci 2010, 11 (5), 316-328. https://doi.org/10.1038/nrn2836.

(130) Rodriguez-Raecke, R.; Niemeier, A.; et al. Brain Gray Matter Decrease in Chronic Pain Is the Consequence and Not the Cause of Pain. The Journal of Neuroscience 2009, 29 (44), 13746-13750.https://doi.org/10.1523/JNEUROSCI.3687-09.2009.

(131) Rothstein, T. L. Cortical Grey Matter Volume Depletion Links to Neurological Sequelae in Post COVID-19 “Long Haulers”. BMC Neurol 2023, 23 (1), 22.https://doi.org/10.1186/s12883-023-03049-1.

(132) Garzón, B.; Lövdén, M.; et al. Role of Dopamine and Gray Matter Density in Aging Effects and Individual Differences of Functional Connectomes. Brain Struct Funct 2021, 226 (3), 743-758. https://doi.org/10.1007/s00429-020-02205-4.

(133) Lai, C.-H.; Wu, Y.-T.; et al. Gray Matter Increases in Fronto-Parietal Regions of Depression Patients with Aripiprazole Monotherapy. Medicine 2016, 95 (34), e4654. https://doi.org/10.1097/MD.0000000000004654.

(134) Moore, T. L.; Young, D. A.; et al. The Effects of a Novel Non-Catechol Dopamine Partial Agonist on Working Memory in the Aged Rhesus Monkey. Front Aging Neurosci 2021, 13. https://doi.org/10.3389/fnagi.2021.757850.

(135) Kozak, R.; Kiss, T.; et al. Characterization of PF-6142, a Novel, Non-Catecholamine Dopamine Receptor D1 Agonist, in Murine and Nonhuman Primate Models of Dopaminergic Activation. Front Pharmacol 2020, 11.https://doi.org/10.3389/fphar.2020.01005.

(136) Vita, A.; De Peri, L.; et al. Progressive Loss of Cortical Gray Matter in Schizophrenia: A Meta-Analysis and Meta-Regression of Longitudinal MRI Studies. Transl Psychiatry 2012, 2 (11), e190. https://doi.org/10.1038/tp.2012.116.

(137) Fountoulakis, K. N.; Stahl, S. M. The Effect of First-and Second-Generation Antipsychotics on Brain Morphology in Schizophrenia: A Systematic Review of Longitudinal Magnetic Resonance Studies with a Randomized Allocation to Treatment Arms. Journal of Psychopharmacology 2022, 36 (4), 428-438.https://doi.org/10.1177/02698811221087645.

(138) Chen, S.; Owens, G. C.; et al. Dopamine Inhibits Mitochondrial Motility in Hippocampal Neurons. PLOS One 2008, 3 (7), e2804.https://doi.org/10.1371/journal.pone.0002804.

(139) Davidson, M. Effects of the D-1 Agonist SKF-38393 Combined With Haloperidol in Schizophrenic Patients. Arch Gen Psychiatry 1990, 47 (2), 190. https://doi.org/10.1001/archpsyc.1990.01810140090014.

(140) Riesenberg, R.; Werth, J.; et al. PF-06649751 Efficacy and Safety in Early Parkinson's Disease: A Randomized, Placebo-Controlled Trial. Ther Adv Neurol Disord 2020, 13, 175628642091129. https://doi.org/10.1177/1756286420911296.

1. A method for treating acute COVID-19 or sequelae of acute COVID-19 infection in a patient, comprising administering to the patient an effective amount of a selective dopamine receptor 1 (D₁R) partial agonist or an angiotensin receptor blocker (ARB) that crosses the blood-brain barrier, or a combination thereof.

2. The method of claim 1, wherein the D₁R partial agonist is selected from at least one of the group consisting of SKF38393, SKF75670, SKF77434, fenoldopam, SKF83959, chloro-APB, SKF81297, SKF82957, SKF83822, SCH-39166, PF-4211, PF-8294, PF-2334, PF-6142, PF-1119, PF-06649751, and ecopipam.

3. The method of claim 1, wherein the D₁R partial agonist is SKF38393 or PF-06649751.

4. The method of claim 1, wherein the ARB is candesartan or telmisartan.

5. The method of claim 1, wherein the combination is administered in a unit dosage form for oral administration.

6. The method of claim 5, wherein the unit dosage form is selected from the group consisting of a tablet, a capsule, a liquid for oral administration and a powder for dissolution or suspension in a beverage.

7. The method of claim 1, wherein a kit is provided having separate oral dosage forms of a D₁R partial agonist and an ARB for administration to the patient.

8. A composition for treating acute COVID-19 or sequelae of acute COVID-19infection in a patient, comprising at least one unit dosage form for oral administration containing a selective dopamine receptor 1 (D₁R) partial agonist or an angiotensin receptor blocker (ARB) that crosses the blood-brain barrier or a combination thereof.

9. The composition of claim 8, wherein the D₁R agonist is selected from at least one of the group consisting of SKF38393, SKF75670, SKF77434, fenoldopam, SKF83959, chloro-APB, SKF81297, SKF82957, SKF83822, SCH-39166, PF-4211, PF-8294, PF-2334, PF-6142, PF-1119 and PF-06649751.

10. The composition of claim 8, wherein the D₁R partial agonist is SKF38393 or PF-06649751.

11. The composition of claim 8, wherein the ARB is candesartan or telmisartan.

12. The composition of claim 8, wherein each unit dosage form is selected from the group consisting of a tablet, a capsule, and a powder suitable for dissolving in a liquid.

13. The composition of claim 12, further comprising an excipient selected from at least one of the group consisting of binders, fillers, disintegrants, glidants, lubricants and flavoring agents, or a combination thereof.
